SEQUENCE LISTING (19 sequences)

SEQ ID NO: 1 - DNA, 743 bp, Homo sapiens
  ref <301>: AUTHORS: Romkes, M., Faletto, M.B., Blaisdell, J.A., Raucy, J.L. and Goldstein, J.A.
  ref <302>: TITLE: Cloning and expression of complementary DNAs for multiple members of the human cytochrome P450IIC subfamily
  ref <303>: JOURNAL: Biochemistry
  ref <304>: VOLUME: 30
  ref <305>: ISSUE: 13
  ref <306>: PAGES: 3247-3255
  ref <307>: DATE: 1991
  ref <308>: DATABASE ACCESSION NUMBER: L16878
  ref <309>: DATABASE ENTRY DATE: 1999-02-08
  ref <313>: RELEVANT RESIDUES IN SEQ ID NO: (1)..(743)
  ref <308>: DATABASE ACCESSION NUMBER: L16878
  ref <309>: DATABASE ENTRY DATE: 1999-02-08
  ref <313>: RELEVANT RESIDUES IN SEQ ID NO: (1)..(743)
tcagaaatatttgaagcctgtgtggctgaataaaagcatacaaatacaatgaaaatatcatgctaaatcaggcttagcaaatggacaaaatagtaacttcgtttgctgttatctctgtctactttcctagctctcaaaggtctatggccctgtgttcactctgtattttggcctgaaacccatagtggtgctgcatggatatgaagcagtgaaggaagccctgattgatcttggagaggagttttctggaagaggcattttcccactggctgaaagagctaacagaggatttggtaggtgtgcatgtgcctgtttcagcatctgtcttggggatggggaggatggaaaacagagacttacagagctcctcgggcagagcttggcccatccacatggctgcccagtgtcagcttcctctttcttgcctgggatctccctcctagtttcgtttctcttcctgttaggaattgttttcagcaatggaaagaaatggaaggagatccggcgtttctccctcatgacgctgcggaattttgggatggggaagaggagcattgaggaccgtgttcaagaggaagcccgctgccttgtggaggagttgagaaaaaccaagggtgggtgaccctactccatatcactgaccttactggactactatcttctctactgacattcttggaaacatttcaggggtggccatatctttcattatgagtctggttgttagctcatgtgaagcgggg

SEQ ID NO: 2 - DNA, 323 bp, Homo sapiens
  ref <301>: AUTHORS: Romkes, M., Faletto, M.B., Blaisdell, J.A., Raucy, J.L. and Goldstein, J.A.
  ref <302>: TITLE: Cloning and expression of complementary DNAs for multiple members of the human cytochrome P450IIC subfamily
  ref <303>: JOURNAL: Biochemistry
  ref <304>: VOLUME: 30
  ref <305>: ISSUE: 13
  ref <306>: PAGES: 3247-3255
  ref <307>: DATE: 1991
  ref <308>: DATABASE ACCESSION NUMBER: L16881
  ref <309>: DATABASE ENTRY DATE: 1999-02-08
  ref <313>: RELEVANT RESIDUES IN SEQ ID NO: (1)..(323)
  ref <308>: DATABASE ACCESSION NUMBER: L16881
  ref <309>: DATABASE ENTRY DATE: 1999-02-08
  ref <313>: RELEVANT RESIDUES IN SEQ ID NO: (1)..(323)
cccctgaattgctacaacaaatgtgccatttttctccttttccatcagtttttacttgtgtcttatcagctaaagtccaggaagagattgaacgtgtgattggcagaaaccggagcccctgcatgcaagacaggagccacatgccctacacagatgctgtggtgcacgaggtccagagataccttgaccttctccccaccagcctgccccatgcagtgacctgtgacattaaattcagaaactatctcattcccaaggtaagtttgtttctcctacactgcaactccatgttttcgaagtcccaaattcatagtatcattttt

SEQ ID NO: 3 - DNA, 394 bp, Homo sapiens
  ref <301>: AUTHORS: Romkes, M., Faletto, M.B., Blaisdell, J.A., Raucy, J.L. and Goldstein, J.A.
  ref <302>: TITLE: Cloning and expression of complementary DNAs for multiple members of the human cytochrome P450IIC subfamily
  ref <303>: JOURNAL: Biochemistry
  ref <304>: VOLUME: 30
  ref <305>: ISSUE: 13
  ref <306>: PAGES: 3247-3255
  ref <307>: DATE: 1991
  ref <308>: DATABASE ACCESSION NUMBER: L16880
  ref <309>: DATABASE ENTRY DATE: 1999-02-08
  ref <313>: RELEVANT RESIDUES IN SEQ ID NO: (1)..(394)
  ref <308>: DATABASE ACCESSION NUMBER: L16880
  ref <309>: DATABASE ENTRY DATE: 1999-02-08
  ref <313>: RELEVANT RESIDUES IN SEQ ID NO: (1)..(394)
aaaaatttccccatcaagatatacaatatattttatttatatttatagctgtaatttacaaccagagcttggtatatggtatgtatgcttttattaaaatcttttaatttaataaattattgttttctcttagatctgcaataatttttctcctatcattgattacttcccgggaactcacaacaaattacttaaaaacgttgcttttatgaaaagttatattttggaaaaagtaaaagaacaccaagaatcaatggacatgaacaaccctcaggactttattgattgcttcctgatgaaaatggagaaggtaaaatgtaaacaaaagcttagttatgtgactgcttgtgaatttgtgatttgttgactagttctgtgtttactaaggatgttt

SEQ ID NO: 4 - DNA, 27 bp, Artificial
  REVERSE PRIMER
cagtaaggtcagtgatatggagtaggg

SEQ ID NO: 5 - DNA, 26 bp, Artificial
  FORWARD PRIMER
tggggaggatggaaaacagagactta

SEQ ID NO: 6 - DNA, 29 bp, Artificial
  FORWARD PRIMER
cagttacacatttgtgcatctgtaaccat

SEQ ID NO: 7 - DNA, 22 bp, Artificial
  REVERSE PRIMER
ctggagaacacacactgccaga

SEQ ID NO: 8 - DNA, 28 bp, Artificial
  FORWARD PRIMER
ccagagcttggtatatggtatgtatgct

SEQ ID NO: 9 - DNA, 31 bp, Artificial
  REVERSE PRIMER
acacagaactagtcaacaaatcacaaattca

SEQ ID NO: 10 - DNA, 44 bp, Artificial
  ASPE PRIMER
ctatctatacatttacaaacattcgaagaggagcattgaggacc

SEQ ID NO: 11 - DNA, 44 bp, Artificial
  ASPE PRIMER
caaacaaacattcaaatatcaatcgaagaggagcattgaggact

SEQ ID NO: 12 - DNA, 44 bp, Artificial
  ASPE PRIMER
tttacaaatctaatcacactatacgcacgaggtccagagataca

SEQ ID NO: 13 - DNA, 44 bp, Artificial
  ASPE PRIMER
ttcattaaacacaaattctatcacgcacgaggtccagagatacc

SEQ ID NO: 14 - DNA, 44 bp, Artificial
  ASPE PRIMER
  modified_base (40)..(40): I
  misc_feature (40)..(40): n is a, c, g, or t
ttcaattcaaatcaaacacatcatgctggtggggagaagntcaa

SEQ ID NO: 15 - DNA, 45 bp, Artificial
  ASPE PRIMER
  modified_base (41)..(41): I
  misc_feature (41)..(41): n is a, c, g, or t
tacacaaacaatctttcacaatttggctggtggggagaagntcag

SEQ ID NO: 16 - DNA, 44 bp, Artificial
  ASPE PRIMER
cattctttaacaacaattctactagcaggctggtggggagaagg

SEQ ID NO: 17 - DNA, 44 bp, Artificial
  ASPE PRIMER
tacattcaacactcttaaatcaaagcaggctggtggggagaagc

SEQ ID NO: 18 - DNA, 44 bp, Artificial
  ASPE PRIMER
attaaacaactcttaactacacaattcctgatgaaaatggagaa

SEQ ID NO: 19 - DNA, 44 bp, Artificial
  ASPE PRIMER
cacatctaatactttatacaattcttcctgatgaaaatggagag

(19) United States
(12) Patent Application Publication
(10) Pub. No.: US 2009/0099030 A1
(43) Pub. Date: Apr. 16, 2009

(54) Method of detecting mutations in the gene encoding cytochrome P450-2C9

(76) Inventor: Frank Merante, Etobicoke (CA)

Correspondence Address: GOODWIN PROCTER LLP;PATENT ADMINISTRATOR, 53 STATE STREET, EXCHANGE PLACE, BOSTON, MA, 02109-2881, US

(21) Appl. No.: 11/631,200

(22) PCT Filed: Jun. 30, 2005
(86) PCT No.: PCT/CA2005/000998

§ 371 Date: Oct. 20, 2008

Related U.S. Application Data
(60) Provisional application No. 60/583,619, filed on Jun. 30, 2004.

(51) Int. Cl. C40B 30/04; C12Q 1/68

(52) U.S. Cl. 506/9; 435/6

(57) ABSTRACT

The present invention describes a method for the simultaneous identification of two or more mutations located in the gene encoding Cytochrome P450-2C9. Multiplex detection is accomplished using multiplexed tagged allele specific primer extension (ASPE) and hybridization of such extended primers to a probe, preferably an addressable anti-tagged support.

### BACKGROUND OF THE INVENTION

1. Field of the Invention

The present invention relates to methods and kits for the detection of mutations located in the gene encoding Cytochrome P450-2C9.

2. Description of the Prior Art

The CYP2C family is responsible for metabolizing a variety of exogenous and endogenous substrates and approximately 20% of currently prescribed drugs. In particular, CYP2C9 has a central role in the phase 1 metabolism of several medications with a narrow therapeutic index (NTI), the best characterized of which include warfarin (Rettie et al., 1992) and phenylonin (Bajpai et al., 1996).

The human cytochrome P450-2C9 gene spans a region of approximately 55 kilobases and is composed of nine exons (de Morais et al., 1993). The gene resides on chromosome 10 (q24) and is clustered among other closely related 2C-genes in the order: Cen-2C18-2C19-2C9-2C8-Tel (Gray et al., 1995).

The 2C9 variant nomenclature follows that outlined by the Human Cytochrome P450 (CYP) Allele Nomenclature Committee (http://www.imm.ki.se/CYPalleles/). The wildtype allele, representing the most common variant is designated 2C9*1. Other variants are accordingly classified as *2 to *12. For example the 2C9*2 variant represents a cytosine to thymine (C→T) transversion at nucleotide 430. This alters the encoded wildtype amino acid to change from an arginine (Arg) to Cysteine (Cys) at position 144 in the polypeptide (R144C).

The six most common CYP2C9 variants are presented in Table 1.

TABLE 1
Most Common CYP4502C9 Variants

| Variant | Nucleotide Change | Effect |
|---|---|---|
| CYP2C9*1 | None | Wildtype |
| CYP2C9*2 | 430 C→T | R114C |
| CYP2C9*3 | 1075 A→C | I359L |
| CYP2C9*4 | 1076 T→C | I359T |
| CYP2C9*5 | 1080 C→G | D360E |
| CYP2C9*6 | 818delA | Frame shift |

Most of the variants, with the exclusion of 2C9*6, result in reduced enzyme activity which has been verified in heterologous expression systems (Haining et al., 1996; Lee et al., 2002). The 2C9*6 variant results in null enzyme activity due to the frameshift in the polypeptide sequence.

FIG. 1 presents a schematic overview of the most commonly encountered CYP2C9 variants.

Genetic testing can be used to identify individuals at risk for adverse drug reactions based on their genetic profile and allow physicians to alter dosing regimens or choose alternate drugs to reduce the potential risk of an adverse drug reaction. A need exists, however, for a rapid, and accurate test for the detection of specific mutations in the gene encoding CYP2C9. A number of manufacturers, for example Motorola, Genlex, ACLARA, and Nanaogen, produce kits that can be used to detect mutations in the CYP2C family, however, most of these kits only detect the two most common mutations in the gene encoding CYP2C9 (2C9*2 and 2C9*3) and exclude the others.

Multiplex Allele Specific Primer Extension and Solid Support Detection of Mutations

Multiplex allele specific primer extension, and hybridization of extended primers to a solid support is described in the prior art ASPE technology has been generally described in U.S. Pat. No. 4,851,331. The technology is designed to identify the presence or absence of specific polymorphic sites in the genome.

Multiplex ASPE in conjunction with hybridization to a support for mutation detection can be described generally as follows:

1) Amplifying regions of DNA comprising polymorphic loci utilizing a multiplexed, PCR.

2) Allele specific extension of primers wherein the amplified regions of DNA serve as target sequences for the allele specific extension. Extension primers that possess a 3′ terminal nucleotide which form a perfect match with the target sequence are extended to form extension products. Modified nucleotides are incorporated into the extension product, such nucleotides effectively labelling the extension products for detection purposes. Alternatively, an extension primer may instead comprise a 3′ terminal nucleotide which forms a mismatch with the target sequence. In this instance, primer extension does not occur.

3) Hybridizing the extension product to a probe on a solid support, such as a microarray, wherein the probe is complementary to the 5′ end of the extension product.

The extension primers used in a methodology as described above, possess unique sequence tags at their 5′ ends. For example, the sequence tags may allow the extension products to be captured on a solid support.

Variations of the above technology have been described, for example, in U.S. Pat. No. 6,287,778 and PCT Application (WO 00/47766).

It is an object of the present invention to provide a cost effective, rapid, and accurate method for the detection of variants in the gene encoding CYP2C9.

### SUMMARY OF THE INVENTION

In one embodiment, the present invention provides a method for detecting the presence or absence of variants in a sample selected from the group of variants identified in Table 1, the method comprising the steps of:

Amplifying regions of DNA which may contain the above mentioned variants using one or more PCR primer pairs selected from the group of PCR pairs consisting of SEQ ID NO.: 4 and SEQ ID NO: 5; SEQ ID NO.: 6 and SEQ ID NO: 7, and SEQ ID NO.: 8 and SEQ ID NO: 9.

Hybridizing at least two tagged allele specific extension primers, the allele specific extension primers selected from the group consisting of SEQ ID NO: 10 to SEQ ID NO: 19, to a complementary region of amplified DNA, each tagged allele specific primer having a 3′ portion complementary to a region of the amplified DNA, a 3′ terminal nucleotide complementary to one allele of one of the mutation sites (wild type or mutant) mentioned above, and a 5′ portion complementary to a probe sequence.

Extending tagged ASPE primers, whereby a labelled extension product of the primer is synthesised when the 3′ terminal nucleotide of the primer is complementary to a corresponding nucleotide in the target sequence; no extension product is synthesised when the terminal nucleotide of the primer is not complementary to the corresponding nucleotide in the target sequence.

Hybridizing extension products to a probe and detection of labelled extension products. Detection of a labelled extension product is indicative of the presence of the allele complementary to the 3′-terminal nucleotide of the ASPE primer. In the absence of a labelled extension product, it is determined that the allele corresponding to the 3′ end of the ASPE primer is not present in the sample.

In another embodiment, the present invention provides a method for detecting the presence or absence of nucleotide variants at polymorphic sites in the gene encoding cytochrome P450-2C9, said variants selected from the group consisting CYP2C9*2, CYP2C9*3, CYP2C9*4, CYP2C9*5, and CYP2C9*6, the method comprising the steps of;

a) amplifying regions of DNA containing the variants to form amplified DNA products;

b) hybridizing at least two tagged allele specific extension primers to a complementary target sequence in the amplified DNA products, wherein each tagged allele specific extension primer has a 3′-end hybridizing portion capable of hybridizing to the amplified DNA, and wherein the 3′ end hybridizing portion of the at least two tagged allele specific extension primers comprise a sequence selected from the group consisting of bases and up of SEQ ID NO: 10 to SEQ ID NO: 19, and a 5′-end tag portion complementary to a corresponding probe sequence, the terminal nucleotide of the 3′ end hybridizing portion being either complementary to a suspected variant nucleotide or to the corresponding wild type nucleotide of the site;

c) extending the at least two tagged allele specific extension primers, using labelled nucleotides, if the terminal nucleotide of the 3′ end hybridizing portion is a perfect match to an allele of one of the polymorphic sites in the amplified DNA products;

d) hybridizing the at least two tagged allele specific extension primers to the corresponding probe sequence and detecting the presence of labelled extension products.

In another embodiment, the present invention provides method for detecting the presence or absence of nucleotide variants at polymorphic sites in the gene encoding cytochrome P450-2C9, said variants selected from the group consisting CYP2C9*2, CYP2C9*3, CYP2C9*4, CYP2C9*5, and CYP2C9*6, the method comprising the steps of;

a) amplifying regions of DNA containing the variants to form amplified DNA products;

b) hybridizing at least two tagged allele specific extension primers to a complementary target sequence in the amplified DNA products, wherein the at least two tagged allele-specific extension primers are selected from the group consisting of SEQ ID NO: 10 to SEQ ID NO: 19, each tagged allele specific extension primer having a 3′-end hybridizing portion capable of hybridizing to the amplified DNA, and a 5′-end tag portion complementary to a corresponding probe sequence, the terminal nucleotide of the 3′ end hybridizing portion being either complementary to a suspected variant nucleotide or to the corresponding wild type nucleotide of the site;

c) extending the at least two tagged allele specific extension primers, using labelled nucleotides, if the terminal nucleotide of the 3′ end hybridizing portion is a perfect match to an allele of one of the polymorphic sites in the amplified DNA products;

d) hybridizing the at least two tagged allele specific extension primers to the corresponding probe sequence and detecting the presence of labelled extension products.

In another embodiment the present invention provides a kit for detecting the presence or absence of nucleotide variants at polymorphic sites in the gene encoding cytochrome P450-2C9, said variants selected from the group consisting CYP2C9*2, CYP2C9*3, CYP2C9*4, CYP2C9*5, and CYP2C9*6, said kit comprising a set of at least two tagged allele specific extension primers wherein each tagged allele specific extension primer has a 3′-end hybridizing portion including a 3′ terminal nucleotide being either complementary to a suspected variant nucleotide or to the corresponding wild type nucleotide of one of the polymorphic sites and a 5′-end tag portion complementary to a corresponding probe sequence, and wherein the at least two tagged allele-specific extension primers are selected from the group consisting of SEQ ID NO: 10 to SEQ ID NO: 19.

In another embodiment, the present invention provides a kit for use in detecting the presence or absence of a variant nucleotide in at least two polymorphic sites in the gene encoding cytochrome P450-2C9, said variants selected from the group consisting CYP2C9*2, CYP2C9*3, CYP2C9*4, CYP2C9*5, and CYP2C9*6, said kit comprising a set of PCR amplification primers for amplifying regions of DNA containing the at least two polymorphic sites, said set comprising at least two pairs of PCR primers selected from the group of pairs consisting of:

### SEQ ID NO: 4 and SEQ ID NO: 5, SEQ ID NO: 6 and SEQ ID NO: 7, and SEQ ID NO: 8 and SEQ ID NO: 9.

### BRIEF DESCRIPTION OF THE DRAWINGS

These and other features of the preferred embodiments of the invention will become more apparent in the following detailed description in which reference is made to the appended drawings wherein:

FIG. 1 depicts a schematic overview of the most common variants in the gene encoding CYP2C9.

FIG. 2 depicts a general overview of steps of the present invention.

FIG. 3 presents a gel presenting the amplification of three regions using the pcr primer pairs of the present invention.

FIG. 4 depicts the genotyping of an individual having a CYP2C9 wildtype genotype.

FIG. 5 depicts the genotyping of an individual having a CYP2C9 2C9*2 and 2C9*3 compound heterozygous genotype.

FIG. 6 depicts the genotyping of an individual having a CYP2C9 2C9*3 heterozygous genotype.

FIG. 7 depicts the genotyping of an individual having a CYP2C9 2C9*2 genotype.

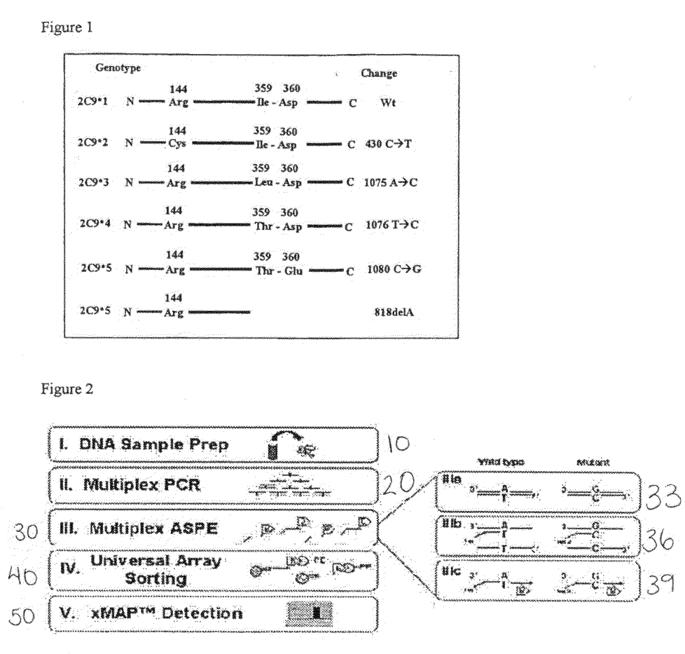

### DESCRIPTION OF THE PREFERRED EMBODIMENTS

The following terms used in the present application will be understood to have the meanings defined below.

The terms “oligonucleotide” and “polynucleotide” as used in the present application refer to DNA sequences being of greater than one nucleotide in length. Such sequences may exist in either single or double-stranded form Examples of oligonucleotides described herein include PCR primers, ASPE primers, and anti-tags.

The term “allele” is used herein to refer to variants of a nucleotide sequence.

The expression “allele specific primer extension (ASPE)”, as used herein, refers to a mutation detection method utilizing primers which hybridize to a corresponding DNA sequence and which are extended depending on the successful hybridization of the 3′ terminal nucleotide of such primer. Amplified regions of DNA serve as target sequences for ASPE primers. ASPE primers include a 3′ end-hybridizing portion which hybridizes to the amplified regions of DNA. ASPE primers that possess a 3′ terminal nucleotide which form a perfect match with the target sequence are extended to form extension products. Modified nucleotides can be incorporated into the extension product, such nucleotides effectively labelling the extension products for detection purposes. Alternatively, an extension primer may instead comprise a 3′ terminal nucleotide which forms a mismatch with the target sequence. In this instance, primer extension does not occur unless the polymerase used for extension inadvertently possesses exonuclease activity or is prone to misincorporation. ASPE primers that possess a 3′ terminal nucleotide which form a perfect match with the target sequence are extended to form extension products. Modified nucleotides can be incorporated into the extension product, such nucleotides effectively labelling the extension 26 products for detection purposes. Alternatively, an extension primer may instead comprise a 3′ terminal nucleotide which forms a mismatch with the target sequence. In this instance, primer extension does not occur unless the polymerase used for extension inadvertently possesses exonuclease activity.

The term “genotype” refers to the genetic constitution of an organism. More 31 specifically, the term refers to the identity of alleles present in an individual. “Genotyping” of an individual or a DNA sample refers to identifying the nature, in terms of nucleotide base, of the two alleles possessed by an individual at a known polymorphic site.

The term “polymorphism”, as used herein, refers to the coexistence of more than one form of a gene or portion thereof.

The term “PCR”, as used herein, refers to the polymerase chain reaction. PCR is a method of amplifying a DNA base sequence using a heat stable polymerase and a pair of primers, one primer complementary to the (+)-strand at one end of the sequence to be amplified and the other primer complementary to the (−) strand at the other end of the sequence to be amplified. Newly synthesized DNA strands can subsequently serve as templates for the same primer sequences and successive rounds of heat denaturation, primer annealing and strand elongation results in rapid and highly specific amplification of the desired sequence. PCR can be used to detect the existence of a defined sequence in a DNA sample.

The term “primer”, as used herein, refers to a short single-stranded oligonucleotide capable of hybridizing to a complementary sequence in a DNA sample. A primer serves as an initiation point for template dependent DNA synthesis. Deoxyribonucleotides can be joined to a primer by a DNA polymerase. A “primer pair” or “primer set” refers to a set of primers including a 5′upstream primer that hybridizes with the complement of the 5′ end of the DNA sequence to be amplified and a 3′ downstream primer that hybridizes with the 3′ end of the DNA sequence to be amplified. The term “PCR primer” as used herein refers to a primer used for a PCR reaction. The term “ASPE primer” as used herein refers to a primer used for an ASPE reaction.

The term “tag” as used herein refers to an oligonucleotide sequence that is coupled to an ASPE primer. The sequence is generally unique and non-complementary to the human genome while being substantially complementary to a probe sequence. The probe sequence may be, for example, attached to a solid support Tags serve to bind the ASPE primers to a probe.

The term “tagged ASPE primer” as used herein refers to an ASPE primer that is coupled to a tag.

The term “anti-tag” or “probe” as used herein refers to an oligonucleotide sequence having a sequence complementary to, and capable of hybridizing to, the tag sequence of an ASPE primer. The “anti-tag” may be coupled to a support.

The term “wild type” or “wt” as used herein refers to the normal, or non-mutated, 32 or functional form of a gene.

The term “homozygous wild-type” as used herein refers to an individual possessing two copies of the same allele, such allele characterized as being the normal and functional form of a gene.

The term “heterozygous” or “HET” as used herein refers to an individual possessing two different alleles of the same gene.

The term “homozygous mutant” as used herein refers to an individual possessing two copies of the same allele, such allele characterized as the mutant form of a gene.

The term “mutant” as used herein refers to a mutated, or potentially non-functional form of a gene.

The present invention was developed in response to a need for a rapid, highly specific, and cost-effective method to genotype individuals susceptible to adverse drug reactions. More specifically, the present invention provides a method for identifying individuals who may have drug metabolism defects resulting from mutations in the CYP2C9 gene.

The present invention provides a novel, multiplex method of detecting multiple mutations located in the gene encoding CYP2C9. Specifically, the methodology can be used for the detection of the presence or absence of two or more mutations selected from the group consisting of the mutations identified in Table 1. In a preferred embodiment, the present invention provides a method of detecting the presence or absence of all the mutations identified in Table 1.

The positive detection of one or more of the mutations identified in Table 1 may be indicative of an individual having a predisposition to compromised enzyme activity.

The present invention is further characterized by a high level of specificity. Such specificity is required in order to ensure that any result generated is a true representation of the genomic target and not simply the result of non-specific interactions occurring between reagents present in reactions. This is especially important for multiplexed DNA-based tests where the numerous sequences present in the reaction mixture, most of which are non-complementary, may interact non-specifically depending on the reaction conditions.

The methodology of the present invention utilizes the combination of multiplex ASPE technology with hybridization of tagged and labelled extension products to probes in order to facilitate detection. Such methodology is suitable for high-throughput clinical genotyping applications.

In one embodiment, the present invention provides a method for detecting the presence or absence of mutations in a sample selected from the group of mutations identified in Table 1, the method comprising the steps of:

Amplifying regions of DNA which may contain the above mentioned mutations.

Hybridizing at least two tagged allele specific extension primers to a complementary region of amplified DNA, each tagged allele specific primer having a 3′ portion complementary to a region of the amplified DNA, a 3′ terminal nucleotide complementary to one allele of one of the mutation sites (wild type or mutant) mentioned above, and a 5′ portion complementary to a probe sequence.

Extending tagged ASPE primers, whereby a labelled extension product of the primer is synthesised when the 3′ terminal nucleotide of the primer is complementary to a corresponding nucleotide in the target sequence; no extension product is synthesised when the terminal nucleotide of the primer is not complementary to the corresponding nucleotide in the target sequence.

Hybridizing extension products to a probe and detection of labelled extension products. Detection of a labelled extension product is indicative of the presence of the allele complementary to the 3′-terminal nucleotide of the ASPE primer. In the absence of a labelled extension product, it is determined that the allele corresponding to the 3′ end of the ASPE primer is not present in the sample.

A general overview of one example of the above-mentioned method is presented in FIG. 2. A DNA sample is first prepared 10 using methods known in the art. Multiplex PCR amplification 20 is conducted in order amplify regions of DNA containing variant sites in the gene encoding cytochrome P450-2C9. A multiplex ASPE reaction 30 is then conducted. By example only, 33 illustrates a wild type and a mutant allele of a gene. At step 36 ASPE primers are hybridized to amplified regions of DNA. If the 3′ terminal nucleotide of an ASPE primer is complementary to a corresponding nucleotide in the target sequence, a labelled extension product is formed 39 as will be described further below. The ASPE may be sorted on an addressable universal sorting array 40 wherein the presence of a labelled extension product may be detected using, for example, xMAP detection 50.

### DNA Sample Preparation

Patient samples can be extracted with a variety of methods known in the art to provide nucleic acid (most preferably genomic DNA) for use in the following method.

### Amplification

In a first step regions of DNA from the gene encoding CYP2C9 containing mutation sites are amplified. The sequences of regions of the CYP 2C9 gene containing the polymorphic sites identified in Table 2 correspond to SEQ ID NO: 1, SEQ ID NO: 2, and SEQ ID NO: 3.

In a preferred embodiment of the present invention, PCR amplification of regions containing mutation sites in the gene encoding CYP2C9 is initiated using at least two pairs of PCR primers selected from the group of primer pairs consisting of: SEQ ID NO.: 4 and SEQ ID NO.: 5, SEQ ID NO.: 6 and SEQ ID NO.: 7, and SEQ ID NO.: 8 and SEQ ID NO.: 9.

The relationships of each pair of primers to the mutation sites listed in Table 2 is presented in Table 3.

TABLE 2
Primer Pairs Used to Amplify Regions
Containing CYP2C9 Mutations

| PCR Primer Pair | Mutations Contained in Amplimer |
|---|---|
| SEQ ID NO: 4 and 5 | 2C9*2 |
| SEQ ID NO: 6 and 7 | 2C9*3, 2C9*4, 2C9*5 |
| SEQ ID NO: 8 and 9 | 2C9*6 |

An individual skilled in the art will recognize that alternate PCR primers could be used to amplify the target polymorphic regions, and deletion and duplication regions, however, in a preferred embodiment the primers listed in Table 2 are selected due to their minimal non-specific interaction with other sequences in the reaction mixture.

### ASPE

The ASPE step of the method of the present invention is conducted using tagged ASPE primers selected from the group of ASPE primers consisting of SEQ ID NO: 10 to SEQ ID NO.: 19.

The ASPE primer set of the present invention has been optimized to ensure high specificity and accuracy of diagnostic tests utilizing such allele specific primers.

Table 3 presents a listing of the ASPE primers used in a preferred embodiment of the present invention. The suffix “wt” indicates an ASPE primer used to detect the wild type form of the gene encoding CYP2C9 at a specific mutation site. The suffix “mut” indicates an ASPE primer used to detect a mutant form of the gene encoding CYP2C9 at a specific mutation site. Bases 1 to 24 of each of SEQ ID NO.: 10 to SEQ ID NO: 19 are the 5′ portions of the ASPE primers that are complementary to specific probe sequences. Although the specific sequences listed in table 3 are preferred, in alternate embodiments of the present invention, it is possible to combine different 5′ portions of the sequences in Table 3 (bases 1 to 24 of SEQ ID NOs: 10 to 19) with different 3′ end hybridizing portions of the sequences in Table 3 (bases 25 and up of SEQ ID NOs: 10 to 19).

The orientation of each of the ASPE primers is also presented in Table 3.

TABLE 3
P450-2C9 ASPE Primer Sequences

| SEQ ID | | |
|---|---|---|
| NO: | Site Detected | Direction |
| 10 | 2C9*2 Wt-C | Forward |
| 11 | 2C9*2 Mut-T | Forward |
| 12 | 2C9*3 Wt-A | Forward |
| 13 | 2C9*3 Mut-C | Forward |
| 14 | 2C9*4 Wt-T | Reverse |
| 15 | 2C9*4 Mut-C (n + 1) | Reverse |
| 16 | 2C9*5 Wt-C | Reverse |
| 17 | 2C9*5 Mut-G | Reverse |
| 18 | 2C9*6 Wt-A | Forward |
| 19 | 2C9*6 Mut-G | Forward |

The 3′ end hybridizing portion of the extension primer is hybridized to the amplified material: Where the 3′ terminal nucleotide of an ASPE primer is complementary to the polymorphic site, primer extension is carried out using a modified nucleotide. Where the 3′ terminal nucleotide of the ASPE primer is not complementary to the polymorphic region, no primer extension occurs.

In one embodiment, labelling of the extension products is accomplished through the incorporation of biotinylated nucleotides into the extension product which may be identified using fluorescent (Streptavidin-Phycoerythrin) or chemiluminescent (Streptavidin-Horseradish Peroxidase) reactions. However, an individual skilled in the art will recognize that other labelling techniques may be utilized. Examples of labels useful for detection include but are not limited to radiolabels, fluorescent labels (e.g. fluorescein and rhodamine), nuclear magnetic resonance active labels, positron emitting isotopes detectable by a positron emission tomography (“PET”) scanner, and chemiluminescers such as luciferin, and enzymatic markers such as peroxidase or phosphatase.

Each ASPE primer used in the methodology as described above, possess a unique sequence tag at their 5′ ends. The sequence tags allow extension products to be detected with a high degree of specificity, for example, through capture on a solid support in order to facilitate detection.

Detection

The tagged 5′ portions of the allele specific primers of the present invention are complementary to probe sequences. Upon hybridization of the allele specific primers to a corresponding probe sequence the presence of extension products can be detected.

In a preferred embodiment, probes used in the methodology of the present invention are coupled to a solid support, for example a ‘universal’ bead-based microarray.

Examples of supports that can be used in the present invention include, but are not limited to, bead based microarrays and 2D glass microarrays. The preparation, use, and analysis of microarrays are well known to persons skilled in the art. (See, for example, Brennan, T. M. et al. (1995) U.S. Pat. No. 5,474,796; Schena, et al. (1996) Proc. Natl. Acad. Sci. 93: 10614-10619; Baldeschweiler et al. (1995), PCT Application WO95/251116; Shalon, D. et al. (1995) PCT application WO95/35505; Heller, R. A. et al. (1997) Proc. Natl. Acad. Sci. 94: 2150-2155; and Heller, M. J. et al. (1997) U.S. Pat. No. 5,605,662.). Detection can be achieved through arrays using, for example, chemiluminescence or fluorescence technology for identifying the presence or absence of specific mutations.

Universal arrays function as sorting tools indirectly detecting the target of interest and are designed to be isothermal and minimally cross-hybridizing as a set. Examples of microarrays which can be used in the present invention include, but should not be limited to, Luminex's® bead based microarray systems, and Metrigenix's™ Flow Thru chip technology.

In one embodiment, for example, Luminex's 100 xMAP™ fluorescence based solid support microarray system is utilized. Anti-tag sequences complementary to the tag regions of the ASPE primers/extension products, described above, are coupled to the surface of internally fluorochrome-color-coded microspheres. An array of anti-tag microspheres is produced, each set of microspheres having its own characteristic spectral address. The mixture of tagged, extended, biotinylated ASPE primers is combined with the array of anti tagged microspheres and is allowed to hybridize under stringent conditions.

In a reaction mixture, a fluorescent reporter molecule (e.g. streptavidin-phycoerythrin) is used to detect labelled extension products which are synthesized when the terminal nucleotide of an ASPE primer is complementary to a corresponding nucleotide in the target sequence.

The reaction mixture, comprising microspheres, extension products etc. is injected into a reading instrument, for example Luminex's 100 xMAP™, which uses microfluidics to align the microspheres in single file. Lasers are used to illuminate the colors both internal to the microspheres, and attached to the surface in the form of extension products hybridized to anti-tag sequences. The Luminex 100 xMAP™, interprets the signal received and identifies the presence of wild type and/or mutant alleles. The presence of the mutant allele of any one or more of the mutations presented in Table 2 may be indicative a predisposition for adverse drug reactions. Software can be provided which is designed to analyze data associated with the specific extension products and anti-tagged microspheres of the present invention.

In another embodiment, the Metrigenix Flow-Thru three dimensional microchannel biochip (Cheek, B. J., Steel A. B., Torres, M. P., Yu, Y., and Yang H. Anal. Chem. 2001, 73, 5777-5783) is utilized for genotyping as known in the art. In this embodiment, each set of microchannels represents a different universal anti-tag population. Anti-tag sequences corresponding to the tag regions of the ASPE primers/extension products, described above, are attached to the inner surface of multiple microchannels comprising a cell. Multiple cells make up a chip. The reaction mixture, including biotinylated extension products flows through the cells in the presence of a chemiluminescent reporter substrate such as streptavidin-horseradish peroxidase. Microarray chips can be imaged using technology known in the art, such as an ORCA-ER CCD (Hamamatsu Photonics K. K., Hamamatsu City, Japan), and imaging software, in order to identify the genotype of an individual.

### Kits

In an additional embodiment, the present invention provides kits for the multiplex detection of mutations in the gene encoding CYP2C9.

A kit that can be used for detection of the mutations of interest may contain the following components including: a PCR primer mix for amplifying regions containing mutation sites of interest (optionally including dNTPs), an ASPE primer mix for generation of labelled extension products (optionally including dNTPs) and a solid support, such as microarray beads, the beads having anti-tags complementary to the tagged regions of the ASPE primers. In addition, an individual skilled in the art would recognize other components which could be included in such kits including, for example, buffers and polymerases.

Kits of the present invention may include PCR primer pairs, ASPE primers, and tagged supports for all the mutations to be detected, or may be customized to best suit the needs of an individual end user. For example, if an end user wishes to detect only four of the mutations in the CYP2C9 gene, a kit can be customized to include only the PCR primer pairs, ASPE primers, and support required for the detection of the desired mutations. As such, the end user of the product can design a kit to match their specific requirements. In addition, the end user can also control the tests to be conducted at the software level when using, for example, a universal bead based-microarray for detection. For example, software can be provided with a kit, such software reading only the beads for the desired mutations or by reporting only the results from the desired mutation data. Similar control of data reporting by software can be obtained when the assay is performed on alternate platforms.

An individual skilled in the art will recognize that although the present method has been described in relation to the specific mutations identified in Table 1, PCR primers and ASPE primers used to detect additional mutations could be included in the above method and kits.

### Example #1

### ASPE/Microarray Detection of Mutations in the Gene Encoding CYP2C9

1) Oligonucleotides

All oligonucleotides were synthesized by Integrated DNA Technologies (Coralville, Iowa). PCR primers were unmodified and were purified by standard desalting procedures. Universal anti-tags (probes) were 3′-C7 amino-modified for coupling to carboxylated microspheres. All anti-tags were reverse phase HPLC-purified. Chimeric ASPE primers which consisted of a 24 mer universal tag sequence 5′ to the allele-specific sequence were also unmodified but were purified by polyacrylamide gel electrophoresis. Following reconstitution, exact oligonucleotide concentrations were determined spectrophotometrically using extinction coefficients provided by the supplier. Reconstituted oligonucleotides were scanned between 200 and 800 nm and absorbance was measured at 260 nm to calculate oligonucleotide concentration.

2) Reagents

Platinum Taq, Platinum Tsp, individual dNTPs and biotin-dCTP were purchased from Invitrogen Corporation (Carlsbad, Calif.). Shrimp alkaline phosphatase and exonuclease I were purchased from USB Corporation (Cleveland, Ohio). Carboxylated fluorescent microspheres were provided by Luminex Corporation (Austin, Tex.): The EDC cross-linker (1-ethyl-3-(3-dimethylaminopropyl) carbodiimide hydrochloride) was purchased from Pierce (Rockford, Ill.). OmniPur reagents including MES (2-(N-morpholino)ethane sulfonic acid), 10% SDS, NaCl, Tris, Triton X-100, Tween-20 and TE buffer were purchased from EM Science (Darrnstadt, Germany). The streptavidin-conjugated phycoerythrin was obtained from Molecular Probes Inc. (Eugene, Oreg.).

3) Genotyping

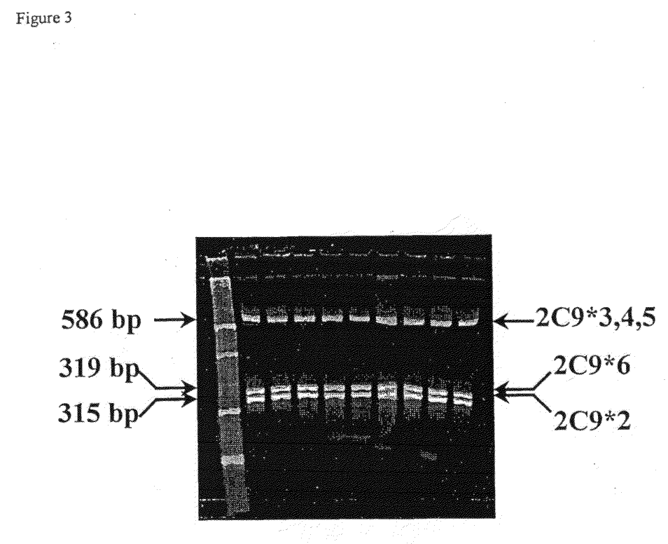

a) MULTIPLEX PCR (3-plex): Multiplex PCR was carried out using 25 ng genomic DNA in a 25 uL final volume. A ‘no target’ PCR negative control was included with each assay run. The reaction consisted of 30 mmol/L Tris-HCl, pH 8.4, 75 mmol/L KCl, 2 mmol/L MgCl₂, 200 umol/L each dNTP, 5 units Platinum Taq and primers at 0.8 umol/L. Samples were cycled in an MJ Research PTC-200 thermocycler (Waterdown, Mass.) with cycling parameters set at 95° C. for 5 minutes followed by 30 cycles at 95° C. for 30 seconds, 58° C. for 30 seconds and 72° C. for 30 seconds. Samples were then held at 72° C. for 5 minutes and kept at 4° C. until use. FIG. 3 depicts a gel presenting the detection of three amplimers obtained using the primer pairs of the present invention.

b) ALLELE-SPECIFIC PRIMER EXTENSION: Prior to the ASPE reaction, each PCR reaction was treated with shrimp alkaline phosphatase (SAP) to inactivate any remaining nucleotides (particularly dCTP) so that biotin-dCTP could be efficiently incorporated during the primer extension reaction. Each PCR reaction was also treated with exonuclease I (EXO) to degrade remaining PCR primers in order to avoid any interference with the tagged ASPE primers and the extension reaction itself. To each 25 uL PCR reaction, 2.0 uL SAP (2.0 units) and 0.5 uL EXO (5 units) were added directly and the sample was vortexed and briefly centrifuged. Samples were then incubated at 37° C. for 30 minutes followed by a 15 minute incubation at 99° C. to inactivate the enzymes. Samples were then added directly to the ASPE reaction.

Multiplex ASPE was carried out using 5 uL of treated PCR product in a final volume of 20 uL. Each reaction consisted of 20 mmol/L Tris-HCl pH 8.4, 50 mmol/L KCl, 1.25 mmol/L MgCl2, 5 mmol/L biotin-dCTP, 5 umol/L each of dATP, dGTP and dTFP, 1.5 units Platinum Tsp and 50 nmol/L ASPE primer pool. The ASPE reactions were incubated at 96° C. for 2 minutes and then subjected to 40 cycles at 94° C. for 30 seconds, 52° C. for 30 seconds and 74° C. for 60 seconds. Reactions were then held at 4° C. until use.

c) BEAD COUPLING: Amino-modified anti-tag sequences were coupled to carboxylated microspheres following Luminex's one-step carbodiimide coupling procedure. Briefly, 5×10⁶ microspheres were combined with 1 nmol NH₂-oligo in a final volume of 50 uL 0.1 mol/L MES, pH 4.5. A 10 mg/mL EDC working solution was prepared just prior to use and 2.5 uL was added to the bead mixture and incubated for 30 minutes. A second 2.5 uL aliquot of freshly prepared EDC was added followed by an additional 30 minute incubation. Following washes in 0.02% (v/v) Tween-20 and 0.1% (w/v) SDS, the anti-tag coupled beads were resuspended in 100 uL TE buffer (10 mmol/L Tris, pH 8.0, 1 mmol/L EDTA). Bead concentrations were determined using a Beckman Coulter Z2 Particle Count and Size Analyzer (Coulter Corp, Miami Fla.).

d) UNIVERSAL ARRAY HYBRIDIZATION: Each hybridization reaction was carried out using approximately 2500 beads of each of the 10 anti-tag bearing bead populations. The beads were combined in hybridization buffer (0.22 mol/L NaCl, 0.11 mol/L Tris, pH 8.0 and 0.088% (v/v) Triton X-100) and 45 uL of the mix were added to each well of an MJ Research 96-well plate (Reno, Nev.). A 5 uL aliquot of each ASPE reaction was then added directly to each well. The samples were then heated to 96° C. for 2 minutes in an MJ Research PTC-100 followed by a one hour incubation at 37° C. Following this incubation, samples were filtered through a 1.2 um Durapore Membrane (Millipore Corp, Bedford, Mass.) and washed once using wash buffer (0.2 mol/L NaCl, 0.1 mol/L Tris, pH 8.0 and 0.08% (v/v) Triton X-100). The beads were then resuspended in 150 uL reporter solution (1 ug/mL streptavidin-conjugated phycoerythrin in wash buffer) and incubated for 15 minutes at room temperature. The reactions were read on the Luminex xMAP. Acquisition parameters were set to measure 100 events per bead population and a 100 uL sample volume. A gate setting was established prior to running the samples and maintained throughout the course of the study.

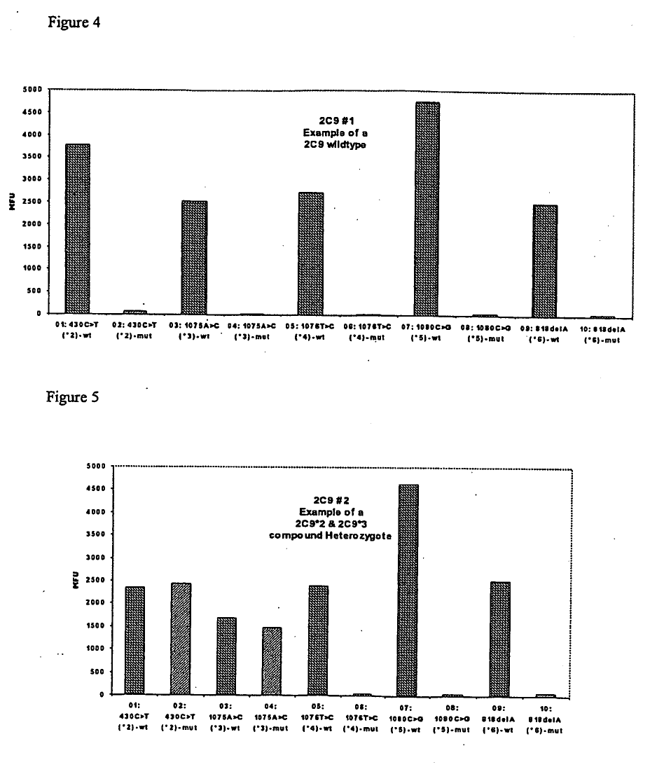

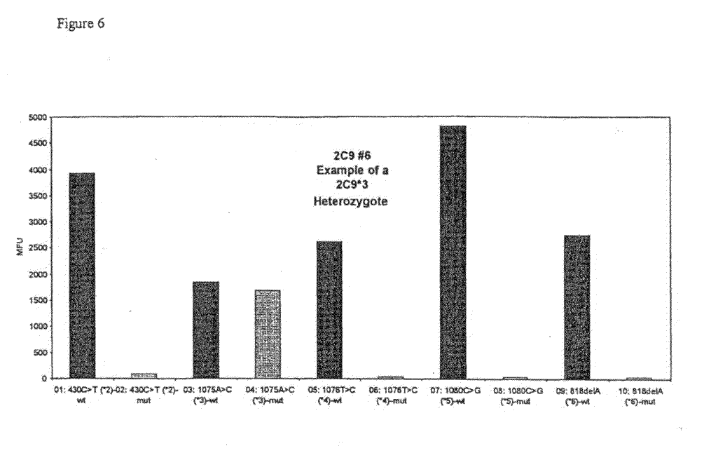

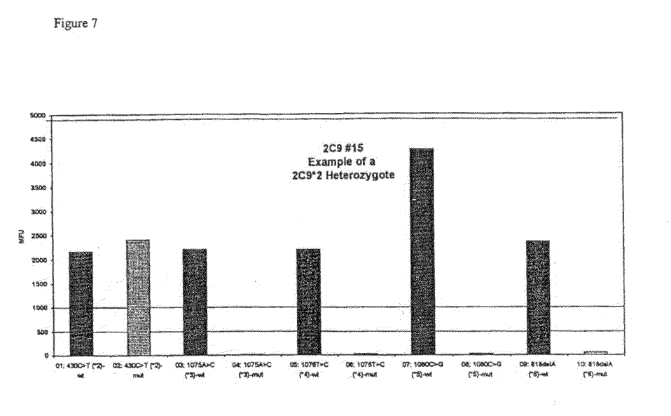

FIGS. 4 to 7 depict a number of results obtained for samples from different individuals using the method of the present invention. FIG. 4 depicts the genotyping of an individual having a CYP2C9 wildtype genotype. FIG. 5 depicts the genotyping of an individual having a CYP2C9 2C9*2 and 2C9*3 compound heterozygous genotype. FIG. 6 depicts the genotyping of an individual having a CYP2C9 2C9*3 heterozygous genotype. FIG. 7 depicts the genotyping of an individual having a CYP2C9 2C9*2 genotype.

All publications, patents and patent applications are herein incorporated by reference in their entirety to the same extent as if each individual publication, patent or patent application was specifically and individually indicated to be incorporated by reference in its entirety

Although the invention has been described with reference to certain specific embodiments, various modifications thereof will be apparent to those skilled in the art without departing from the spirit and scope of the invention as outlined in the claims appended hereto.

The embodiments of the invention in which an exclusive property or privilege is claimed are defined as follows:

1. A method for detecting the presence or absence of nucleotide variants at polymorphic sites in the gene encoding cytochrome P450-2C9, said variants selected from the group consisting CYP2C9*2, CYP2C9*3, CYP2C9*4, CYP2C9*5, and CYP2C9*6, the method comprising the steps of;
a) amplifying regions of DNA containing the variants to form amplified DNA products;
b) hybridizing at least two tagged allele specific extension primers to a complementary target sequence in the amplified DNA products, wherein each tagged allele specific extension primer has a 3′-end hybridizing portion capable of hybridizing to the amplified DNA, and wherein the 3′ end hybridizing portion of the at least two tagged allele specific extension primers comprise a sequence selected from the group consisting of bases 25 and up of SEQ ID NO: 10 to SEQ ID NO: 19, and a 5′-end tag portion complementary to a corresponding probe sequence, the terminal nucleotide of the 3′ end hybridizing portion being either complementary to a suspected variant nucleotide or to the corresponding wild type nucleotide of the site;
c) extending the at least two tagged allele specific extension primers, using labelled nucleotides, if the terminal nucleotide of the 3′ end hybridizing portion is a perfect match to an allele of one of the polymorphic sites in the amplified DNA products;
d) hybridizing the at least two tagged allele specific extension primers to the corresponding probe sequence and detecting the presence of labelled extension products.

2. The method of claim 2 wherein the 5′-end tag portions of the at least two tagged allele specific primers comprises a sequence selected from the group consisting of bases 1 to 24 of SEQ ID NO: 10 to SEQ ID NO: 19.

3. The method of claim 1 wherein the probe sequence is coupled to a solid support.

4. The method of claim 3 wherein the solid support is selected from the group consisting of beads, spectrally coded beads, and a chip based microarray.

5. The method of claim 1 wherein the step of amplifying is conducted by PCR using a set of PCR amplification primers, said set comprising at least two pairs of PCR primers selected from the group of pairs consisting of:
SEQ ID NO: 4 and SEQ ID NO: 5, SEQ ID NO: 6 and SEQ ID NO: 7, and SEQ ID NO: 8 and SEQ ID NO: 9.

6. A method for detecting the presence or absence of nucleotide variants at polymorphic sites in the gene encoding cytochrome P450-2C9, said variants selected from the group consisting CYP2C9*2, CYP2C9*3, CYP2C9*4, CYP2C9*5, and CYP2C9*6, the method comprising the steps of;
a) amplifying regions of DNA containing the variants to form amplified DNA products;
b) hybridizing at least two tagged allele specific extension primers to a complementary target sequence in the amplified DNA products, wherein the at least two tagged allele-specific extension primers are selected from the group consisting of SEQ ID NO: 10 to SEQ ID NO: 19, each tagged allele specific extension primer having a 3′-end hybridizing portion capable of hybridizing to the amplified DNA, and a 5′-end tag portion complementary to a corresponding probe sequence, the terminal nucleotide of the 3′ end hybridizing portion being either complementary to a suspected variant nucleotide or to the corresponding wild type nucleotide of the site;
c) extending the at least two tagged allele specific extension primers, using labelled nucleotides, if the terminal nucleotide of the 3′ end hybridizing portion is a perfect match to an allele of one of the polymorphic sites in the amplified DNA products;
d) hybridizing the at least two tagged allele specific extension primers to the corresponding probe sequence and detecting the presence of labelled extension products.

7. The method of claim 6 wherein the probe sequence is coupled to a solid support.

8. The method of claim 7 wherein the solid support is selected from the group consisting of beads, spectrally coded beads, and a chip based microarray.

9. The method of claim 6 wherein the step of amplifying is conducted by PCR using a set of PCR amplification primers, said set comprising at least two pairs of PCR primers selected from the group of pairs consisting of SEQ ID NO: 4 and SEQ ID NO: 5, SEQ ID NO: 6 and SEQ ID NO: 7, and SEQ ID NO: 8 and SEQ ID NO: 9.

10. A kit for detecting the presence or absence of nucleotide variants at polymorphic sites in the gene encoding cytochrome P450-2C9, said variants selected from the group consisting CYP2C9*2, CYP2C9*3, CYP2C9*4, CYP2C9*5, and CYP2C9*6, said kit comprising a set of at least two tagged allele specific extension primers wherein each tagged allele specific extension primer has a 3′-end hybridizing portion including a 3′ terminal nucleotide being either complementary to a suspected variant nucleotide or to the corresponding wild type nucleotide of one of the polymorphic sites and a 5′-end tag portion complementary to a corresponding probe sequence, and wherein the at least two tagged allele-specific extension primers are selected from the group consisting of SEQ ID NO: 10 to SEQ ID NO: 19.

11. The kit of claim 10 further comprising a set of PCR amplification primers for amplifying regions of DNA containing the polymorphic sites, said set comprising at least two pairs of PCR primers selected from the group of pairs consisting of:
SEQ ID NO: 4 and SEQ ID NO: 5, SEQ ID NO: 6 and SEQ ID NO: 7, and SEQ ID NO: 8 and SEQ ID NO:9.

12. The kit of claim 10 further comprising a set of probes.

13. The kit of claim 12 wherein the set of probes are coupled to a support.

14. A kit for use in detecting the presence or absence of a variant nucleotide in at least two polymorphic sites in the gene encoding cytochrome P450-2C9, said variants selected from the group consisting CYP2C9*2, CYP2C9*3, CYP2C9*4, CYP2C9*5, and CYP2C9*6, said kit comprising a set of PCR amplification primers for amplifying regions of DNA containing the at least two polymorphic sites, said set comprising at least two pairs of PCR primers selected from the group of pairs consisting of:
SEQ ID NO: 4 and SEQ ID NO: 5, SEQ ID NO: 6 and SEQ ID NO: 7, and SEQ ID NO: 8 and SEQ ID NO: 9.

15. The kit of claim 14 further comprising a set of at least two tagged allele specific extension primers wherein each tagged allele specific extension primer has a 3′-end hybridizing portion capable of hybridizing to the amplified DNA, a 5′-end tag portion complementary to a corresponding probe sequence, the terminal nucleotide of the 3′ end hybridizing portion being either complementary to a suspected variant nucleotide or to the corresponding wild type nucleotide of the polymorphic sites.
